(19) United States
(12) Patent Application Publication
(10) Pub. No.: US 2019/0043332 A1
(43) Pub. Date: Feb. 7, 2019

(54) Gas Monitoring System with RFID Gas Micro Sensor

(71) Applicant: David Bublil, Ramat Gan (IL)

(72) Inventor: David Bublil, Ramat Gan (IL)

(21) Appl. No.: 16/071,484

(22) PCT Filed: Jan. 16, 2017
(86) PCT No.: PCT/IL2017/050047

§ 371 Date: Jul. 20, 2018

Related U.S. Application Data
(60) Provisional application No. 62/280,745, filed on Jan. 20, 2016.

(30) Foreign Application Priority Data
Jan. 16, 2017 (IL) 250131

(51) Int. Cl. G08B 21/14; F23N 5/24; G08B 25/10; G01N 33/00; G08B 17/00

(52) U.S. Cl. CPC G08B 21/14; F23N 5/242; G08B 17/00; G01N 33/0004; G08B 25/10

(57) ABSTRACT

A gas monitoring system that includes a gas detector that includes a RFID Gas Micro Sensor and a reader that includes a computer processor and a receiver. The reader and the RFID Gas Micro Sensor have communication program that is designed to enable them to communicate one with the other. The RFID Gas Micro Sensor is designed to be installed on or near a gas tank or a gas pipe. The RFID Gas Micro Sensor is designed to sense a gas leak and transmits information to the reader. The reader is designed to provide alert, or to transfer the information to a control center or to provide an automatic response.

### TECHNICAL FIELD

The present invention refers to a gas monitoring system.

### BACKGROUND ART

Detecting gas leaks in real time and providing an alert and quick response for such leaks are necessary for lifesaving and protecting infrastructures and properties. There are many known systems for detecting gas leaks, alerting and providing response in such cases, but these systems are cumbersome and inefficient in many cases. The present invention provides efficient and innovative systems for detecting, alerting and providing response for gas leaks.

### DESCRIPTION OF THE DRAWINGS

The intention of the drawings attached to the application is not to limit the scope of the invention and its application. The drawings are intended only to illustrate the invention and they constitute only one of its many possible implementations.

FIG. 1 depicts schematically the Gas Detector (1) which based on the RFID Gas Micro Sensor (2) and a smartphone as its reader (3).

FIG. 2 depicts schematically the Gas Monitoring System (100) which includes several Gas Detectors (1), Several Electric Faucets (4), several Power Turbine Generators (6), and a Control Center (101).

FIG. 3 depicts schematically the Gas Volume Detector System (200) which includes a RFID Gas Micro Sensor (2), a reader (3) and a control center (101).

FIG. 4 depicts schematically the Independent Gas Faucet System (300) which includes an electric facet (4) with integral RFID Gas Micro Sensor (2) and a reader (3).

FIG. 5 depicts schematically the Independent Detector (400) which includes a RFID Gas Micro Sensor (2), a reader (3) and an indicator (5).

FIG. 6 depicts schematically the Stop Fire Detection Alert (500) which includes a sensor (501), electronic chip (502), antenna (503), power source (504) and a reader (505).

### THE INVENTION

The systems subject matter of the present patent application based on the active, passive and half-passive Radio Frequency Identification (RFID) Gas Sensor of General Electric company, which may equipped also with self-correction system against fluctuations of ambient temperature (Hereinafter “the RFID Gas Micro Sensor”). This sensor is designed for detection and quantitation of gases. The RFID Gas Micro Sensor is highly accurate and can detect the quantitation of gas of ambient air. It is possible to calibrate the RFID Gas Micro Sensor in advance to determine the kind of gas that it would detect. This sensor is hundreds times smaller, weighs hundreds times less, and uses hundreds times less power than the conventional known gas sensors.

The RFID Gas Micro Sensor can be an active, a passive or half-passive sensor. An active RFID Gas Micro Sensor includes a small battery, transceiver, electronic chip and communication program for enabling the sensor to communicate with a reader. The active RFID Gas Micro Sensor can be sense the air continuously and transmit the information to a reader. The passive RFID Gas Micro Sensor does not include battery and it senses the air and transmits the information, means to be active, when a magnetic field is activated near it. The half-passive RFID Gas Micro Sensor does not include battery but near it located a battery that creates a magnetic field that enable it to senses the air continuously but to transmits the information only when a magnetic field of a reader is activated near it.

The term “gas” in the present patent application refers to variety kinds of gases including liquefied petroleum gas and also to chemical, organic and biological particles in the air including smoke and air pollution. The term RFID refers to Radio Frequency Identification and also to other communications methods such as RF, NFC and others.

The main objective of the present invention is to provide a gas monitoring system (100) that based on a gas detector (1) that includes the RFID Gas Micro Sensor (2) and a reader (3). The reader (3) can be in fact any device that includes computer processor and receiver, such as any standard smartphone or a reader which may be a device that is designed to be function solely as a reader. The reader (3) and the RFID Gas Micro Sensor (2) have communication program for enabling them to communicate one with the other.

The RFID Gas Micro Sensor (2) is designed to be installed in any place that the user may access that a gas leak may occur. When the RFID Gas Micro Sensor (2) senses a gas leak it transmit the information to the reader (3), and then the reader (3) can provide alert, to transfer the information to control center (101) or to provide automatic response to the situation. The alert can be provided directly by the reader (3) or by any secondary instrument.

The Gas Monitoring System (100) includes the gas detector (1) that includes the RFID Gas Micro Sensor (2) and a reader (3). The Gas Monitoring System (100) also may include a control center (101) mainly when the system (100) includes several gas detectors (1).

The Gas Monitoring System (100) also may include electric faucets (4) that can close and open pipes. The electric faucet (4) can be wired directly to the control system (101) and to electric source or wireless and therefore includes receiver for receiving the relevant command and battery. The control center (101) of the system (100) can send commands for closing and opening the relevant electric faucets (4) along the pipes as a response to detection of gas leak. Such commands can be control by a person or can be automatic response for the gas detection.

The Gas Monitoring System (100) may include one gas detector (1), for example, near a stove a home or many gas detectors (1) and electric valves (4) which may be installed along the pipe and sensitive places in a huge plant.

The Gas Monitoring System (100) may also include a Power Turbine Generator (6) that employed the speed of the gas to generate electric power for the Gas Monitoring System (100) or for its components.

Another embodiment of the present invention is the Gas Volume Detector System (200) that based on the RFID Gas Micro Sensor (2). It is possible to calibrate the RFID Gas Micro Sensor in advance to design it to determine the volume of the gas in a tank and therefore by installing it inside a tank it can transmit to the reader (3) or to the control center (101) the information regarding the volume of the gas in that tank. An algorithm using the information regarding the state of the opening and closing position of the tank can determine whether the gas is consumed in the ordinary way or it is leaked out and consequently to provide alert regarding such leak.

Another embodiment of the present invention is the Independent Gas Faucet System (300) which includes an electric facet (4) with integral RFID Gas Micro Sensor (2) and a reader (3). This system (300) can be installed on along a pipe or instead of the standard faucet and when the RFID Gas Micro Sensor (2) detects a gas then the Independent Gas Faucet System (300) closes itself.

Another embodiment of the present invention is the Independent Gas Detector (400) which includes the RFID Gas Micro Sensor (2), the reader (3) and indicator (5) which may be buzzer, lights or any other means for indication information. This Independent Gas Detector (400) may be used for variety of situations. For example, it can be used by sportsman to know the level of the pollution while doing sport in the park, and for such implementation the RFID Gas Micro Sensor (2) will be designed to detect the relevant gases to consist the pollution. Another example, is when the Independent Gas Detector (400) may be used for fireman to enable them to determine the conditions of the air in the place they exercise their activities. Due to the size of the RFID Gas Micro Sensor (2) the Independent Gas Detector (400) can be worn as a bracelet.

The Gas Monitoring System (100) may be used for personal simple mission, for example for detecting a leak of gas in a car or in a room. In such case, the Gas Monitoring System (100) will be based on a gas detector (1) that includes the RFID Gas Micro Sensor (2) and employing the smartphone of the user as a reader (3). On the other hand, the Gas Monitoring System (100) may be used for complicated and huge missions, for example, for monitoring and controlling big plants. In such case, the Gas Monitoring System (100) will be based on big amount of Gas Detectors (3), electric faucets (4), Power Turbine Generators (6) and control center (101).

Another embodiment of the present invention is the Stop Fire Detection Alert (500) which includes a sensor (501) that is designed to recognize fire nearby, electronic chip (502), antenna (503) and power source (504) which may be a battery of a wire connection to outer power source. The Stop Fire Detection Alert (500) is designed to be located or installed near a fire place, such as the gas stove and when it detects a fire and after that detects that the fire is stopped then it alerts or send an alert to the reader (505) and the user can response. In case the user close the gas and the fire, then he or she can ignore that alert but if the fire stopped by itself, then we can assume that the gas is continuing to stream and the user will response accordingly.

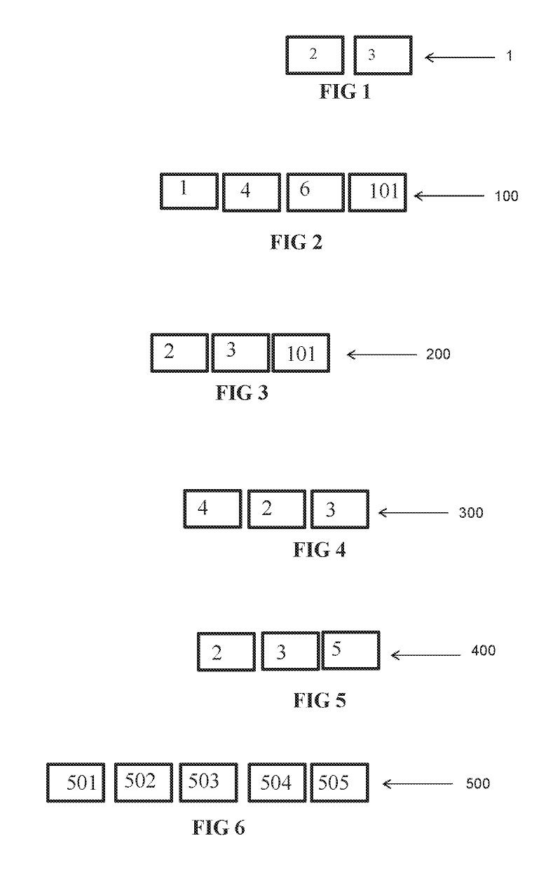

FIG. 1 depicts schematically the Gas Detector (1) which based on the RFID Gas Micro Sensor (2) and a smartphone as its reader (3).

FIG. 2 depicts schematically the Gas Monitoring System (100) which includes several Gas Detectors (1), Several Electric Faucets (4), several Power Turbine Generators (6), and a Control Center (101).

FIG. 3 depicts schematically the Gas Volume Detector System (200) which includes a RFID Gas Micro Sensor (2), a reader (3) and a control center (101).

FIG. 4 depicts schematically the Independent Gas Faucet System (300) which includes an electric facet (4) with integral RFID Gas Micro Sensor (2) and a reader (3).

FIG. 5 depicts schematically the Independent Detector (400) which includes a RFID Gas Micro Sensor (2), a reader (3) and an indicator (5).

FIG. 6 depicts schematically the Stop Fire Detection Alert (500) which includes a sensor (501), electronic chip (502), antenna (503), power source (504) and a reader (505).

What is claimed is:

1. A gas monitoring system, comprising: a gas detector and a reader; wherein the gas detector includes a RFID Gas Micro Sensor; wherein the reader includes computer processor and receiver; wherein the reader and the RFID Gas Micro Sensor have communication program that is designed to enable them to communicate one with the other; wherein the RFID Gas Micro Sensor is designed to be installed on or near a gas tank or a gas pipe; wherein the RFID Gas Micro Sensor is designed to sense a gas leak and transmits information to the reader; wherein the reader is designed to provide alert, or to transfer the information to a control center or to provide an automatic response.

2. The gas monitoring system according to claim 1 that further includes an electric faucet that is designed to close and open gas pipes.

3. The gas monitoring system according to claim 2 wherein said electric faucet includes a receiver for receiving command and a battery.

4. The gas monitoring system according to claim 1 that further includes a power turbine generator that employed a speed of gas in a pipe to generate electric power for said Gas Monitoring System.

5. A Gas Volume Detector System that includes a RFID Gas Micro Sensor (2) that is calibrated and designed to determine volume of a gas in a tank.

6. An Independent Gas Faucet System, comprising: an electric facet with integral RFID Gas Micro Sensor and a reader.

7. An Independent Gas Detector, comprising: a RFID Gas Micro Sensor, a reader and indicator.

8. A Stop Fire Detection Alert, comprising: a sensor that is designed to recognize fire nearby, an electronic chip, an antenna and a power source; wherein said Stop Fire Detection Alert is designed to be located or installed near a fire place, and when it detects a fire and after that detects that the fire is stopped it alerts or send an alert to a reader and whereby enabling a user to response.
